(19) United States
(12) Patent Application Publication
(10) Pub. No.: US 2025/0123164 A1
(43) Pub. Date: Apr. 17, 2025

(54) STRETCHABLE ION-GEL SENSOR AND METHOD OF PREPARING THE SAME

(71) Applicant: POSTECH RESEARCH AND BUSINESS DEVELOPMENT FOUNDATION, Pohang-si (KR)

(72) Inventors: Unyong JEONG, Pohang-si (KR); Hyeongseok CHOI, Pohang-si (KR)

(73) Assignee: POSTECH RESEARCH AND BUSINESS DEVELOPMENT FOUNDATION, Pohang-si (KR)

(21) Appl. No.: 18/731,062

(22) Filed: May 31, 2024

(30) Foreign Application Priority Data
Oct. 17, 2023 (KR) 10-2023-0138189

(51) Int. Cl. G01L 1/22; A61L 27/44; A61L 27/60

(52) U.S. Cl. CPC G01L 1/2287; A61L 27/446; A61L 27/60; A61L 2420/02; A61L 2420/04; A61L 2420/08

(57) ABSTRACT

A stretchable ion-gel sensor and a method of preparing the same are provided. The stretchable ion-gel sensor includes an ion-gel layer, an upper electrode layer disposed on a top surface of the ion-gel layer, and a lower electrode layer disposed on a bottom surface of the ion-gel layer, wherein each of the upper electrode layer and the lower electrode layer includes a first metal particle/polymer layer and a second metal particle/polymer layer.

### CROSS-REFERENCE TO RELATED APPLICATION

This application claims the benefit of Korean Patent Application No. 10-2023-0138189, filed on Oct. 17, 2023, in the Korean Intellectual Property Office, the entire disclosure of which is incorporated herein by reference for all purposes.

### BACKGROUND

### 1. Field of the Invention

One or more embodiments relate to a stretchable ion-gel sensor and a method of preparing the stretchable ion-gel sensor.

### 2. Description of the Related Art

When artificial electronic skins are developed in fields such as healthcare or robotics, electrolyte-based systems (ion gels or ionic gels) are widely used. Electrolyte-based systems are based on inherent characteristics of electrolytes and characteristics at electrolyte-electrode interfaces. When characteristics of electrolytes or characteristics of electrode interfaces change due to external stimulation, such change appears as a change in electrical signals. In addition, various biocompatible synthetic electrodes are being developed to develop neural electrodes for analyzing biological signals in human bodies. To stably maintain characteristics of electrodes despite large or small mechanical movements of organ tissues, a stretching-insensitive electrode model needs to be implemented.

However, due to electrical characteristics of electrolyte-based artificial electronic skins, it is difficult to reliably recognize various types of external stimuli at the same time. For example, when a temperature stimulus and a mechanical stretching stimulus are simultaneously applied, ionic resistance components change due to both the temperature stimulus and the mechanical stretching stimulus, and accordingly, a complex signal processing method such as separating of measurement frequencies is required. Conventional technologies that developed stretching-insensitive nerve electrodes introduced a complex hierarchical structure to minimize effects of mechanical stimuli. Because the complexity of the structure is disadvantageous for application to all microstructures, it is necessary to have the structure as simple as possible.

The above description is information the inventor(s) acquired during the course of conceiving the present disclosure, or already possessed at the time, and is not necessarily art publicly known before the present application was filed.

### SUMMARY

One or more embodiments provide a stretchable ion-gel sensor that may adjust a stretching reactivity through a composite electrode with a simple structure, and a method of preparing the stretchable ion-gel sensor.

However, goals to be achieved are not limited to those described above, and other goals not mentioned above can be clearly understood by one of ordinary skill in the art from the following description.

According to an aspect, there is provided a stretchable ion-gel sensor including an ion-gel layer, an upper electrode layer disposed on a top surface of the ion-gel layer, and a lower electrode layer disposed on a bottom surface of the ion-gel layer, wherein each of the upper electrode layer and the lower electrode layer includes a first metal particle/polymer layer, and a second metal particle/polymer layer.

In an embodiment, the ion-gel layer may include a mixture of an ionic liquid and a polymer binder.

In an embodiment, the ionic liquid may include at least one of 1-ethyl-3-methylimidazolium bis(trifluoromethylsulfonyl)imide ([EMIM][TFSI]), 1-butyl-3-methylimidazolium bis(trifluoromethylsulfonyl)imide ([BMIM][TFSI]), 1-butyl-3-methylimidazolium hexafluorophosphate ([BMIM][PF₆]), 1-ethyl-3-methylimidazolium tetrafluoroborate ([EMIM][BF₄]), 1-butyl-3-methylimidazolium tetrafluoroborate

([BMIM][BF₄]), 1-butyl-1-methylpyrrolidinium bis(trifluoromethylsulfonyl)imide ([BMPYR][TFSI]), 1-butyl-1-methylpyrrolidinium tris(pentafluoroethyl)trifluorophosphate ([BMPYR][FAP]), 1-ethyl-3-methylimidazolium tris(pentafluoroethyl)trifluorophosphate ([EMIM][FAP]), 1-ethyl-3-methylimidazolium bis(fluorosulfonyl)imide ([EMIM][FSI]), and ethyl-dimethyl-propylammonium bis(trifluoromethylsulfonyl)imide ([EDMPA][TFSI]).

In an embodiment, the polymer binder may include at least one of poly(vinylidene fluoride-co-hexafluoropropylene) (P(VDF-HFP)), poly(styrene-block-ethylene oxide-block-styrene) (PS-PEO-PS), and poly(styrene-block-methyl methacrylate-block-styrene) (PS-PMMA-PS).

In an embodiment, the first metal particle and the second metal particle may each have a diameter of 5 micrometers (μm) to 20 μm.

In an embodiment, the first metal particle and the second metal particle may each include at least one of gold (Au), silver (Ag), platinum (Pt), palladium (Pd), copper (Cu), cobalt (Co), zirconium (Zr), zinc (Zn), titanium (Ti), and tin (Sn).

In an embodiment, the first metal particle of the upper electrode layer may be in an amount of 75% by weight (wt %) to 80 wt % in the first metal particle/polymer layer.

In an embodiment, the second metal particle of the upper electrode layer may be in an amount of 67 wt % to 80 wt % in the second metal particle/polymer layer.

In an embodiment, the first metal particle of the lower electrode layer may be in an amount of 75 wt % to 80 wt % in the first metal particle/polymer layer.

In an embodiment, the second metal particle of the lower electrode layer may be in an amount of 67 wt % to 80 wt % in the second metal particle/polymer layer.

In an embodiment, the polymer layer of the upper electrode layer and the polymer layer of the lower electrode layer each may include at least one of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

In an embodiment, each of the upper electrode layer and the lower electrode layer May further include an elastic substrate.

In an embodiment, the elastic substrate may include at least one of polydimethylsiloxane (PDMS), a fluoroelastomer, a poly(vinylidene fluoride-co-hexafluoropropylene) copolymer, thermosetting polyurethane, silicone, Ecoflex, and Dragon skin.

In an embodiment, when an amount of metal particles in the first metal particle/polymer layer is less than 75 wt %, the stretchable ion-gel sensor may have an impedance of 10⁶ ohms (Ω) to 10⁷ Ω in a frequency range of 10⁰ hertz (Hz) to 10² Hz. When the amount of the metal particles in the first metal particle/polymer layer is greater than or equal to 75 wt % and less than or equal to 80 wt %, the impedance of 10⁶ Ω to 10⁷ Ω may be maintained in the frequency range of 100 Hz to 102 Hz. When the amount of the metal particles in the first metal particle/polymer layer exceeds 80 wt %, the impedance may be in a range of 10⁶ Ω to 10⁹ Ω in the frequency range of 100 Hz to 102 Hz.

According to another aspect, there is provided a method of preparing a stretchable ion-gel sensor, the method including separately preparing an upper electrode layer and a lower electrode layer, forming an ion-gel layer on each of the upper electrode layer and the lower electrode layer, and arranging the ion-gel layer of the upper electrode layer and the ion-gel layer of the lower electrode layer to face each other and performing annealing.

In an embodiment, the separately preparing of the upper electrode layer and the lower electrode layer may include forming a sacrificial layer on a substrate, forming a first metal particle/polymer layer and a second metal particle/polymer layer after placing a pattern mask on the sacrificial layer, annealing the first metal particle/polymer layer and the second metal particle/polymer layer after removing the pattern mask, forming an elastic substrate by coating the annealed first metal particle/polymer layer and the annealed second metal particle/polymer layer with an elastic polymer and curing the elastic polymer, and separating the substrate by removing the sacrificial layer.

In an embodiment, the forming of the sacrificial layer on the substrate may include applying at least one solution among polyacrylic acid (PAA), polyvinylpyrrolidone (PVP), dextran, poly(methyl methacrylate) (PMMA), and poly(vinyl alcohol) (PVA) onto the substrate by spin coating, and annealing the substrate at a temperature of 80° C. to 150° C. for 10 minutes to 60 minutes, to form the sacrificial layer.

In an embodiment, each of the first metal particle and the second metal particle may include at least one of gold (Au), silver (Ag), platinum (Pt), palladium (Pd), copper (Cu), cobalt (Co), zirconium (Zr), zinc (Zn), titanium (Ti), and tin (Sn).

In an embodiment, the polymer layer of the upper electrode layer and the polymer layer of the lower electrode layer may each include at least one of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

In an embodiment, the forming of the first metal particle/polymer layer and the second metal particle/polymer layer may include forming the first metal particle/polymer layer by applying a first metal/polymer resin ink, obtained by mixing a first metal particle and a polymer resin, by blade coating, plasma-treating a surface of the first metal particle/polymer layer, and forming the second metal particle/polymer layer by applying a second metal/polymer resin ink, obtained by mixing a second metal particle and a polymer resin, onto the plasma-treated surface of the first metal particle/polymer layer by blade coating.

In an embodiment, the plasma-treating of the surface may include performing an oxygen plasma treatment in a power range of 80 watts (W) to 120 W at an oxygen flow rate of 30 standard cubic centimeters per minute (sccm) to 50 sccm for 1 second to 300 seconds.

In an embodiment, the annealing of the first metal particle/polymer layer and the second metal particle/polymer layer may include performing annealing under a vacuum condition in a temperature range of 100° C. to 200° C. for 1 hour to 6 hours.

In an embodiment, the forming of the elastic substrate may include applying the elastic polymer onto the first metal particle/polymer layer and the second metal particle/polymer layer by spin coating, and performing annealing in a temperature range of 20° C. to 100° C. for 30 minutes to 300 minutes.

In an embodiment, the separating of the substrate by removing the sacrificial layer may include immersing the substate in deionized water in a temperature range of 20° C. to 100° C. for 30 minutes to 300 minutes and removing the sacrificial layer, to separate the substrate.

In an embodiment, the arranging of the ion-gel layer of the upper electrode layer and the ion-gel layer of the lower electrode layer to face each other and performing annealing may be performed at a temperature of 80° C. to 150° C. for 1 minute to 60 minutes.

Additional aspects of embodiments will be set forth in part in the description which follows and, in part, will be apparent from the description, or may be learned by practice of the disclosure.

According to embodiments, a stretchable ion-gel sensor may simply change a stretching reactivity in various directions by adjusting a composition ratio of metal particles in a first metal particle/polymer layer and a second metal particle/polymer layer with a simple structure.

According to embodiments, a stretchable ion-gel sensor may be applied to artificial electronic skins compatible with the Internet of Things (IoT), skin-attachable flexible sensors, electrocardiogram monitoring medical devices, smart wearable devices, bioelectrodes for analysis of nerve stimulation, and electronic skins for robots. In addition, the stretchable ion-gel sensor may be applied to artificial electronic skin, skin-attachable medical devices, biosignal analysis devices, and robot skins in the future.

According to embodiments, by a method of preparing a stretchable ion-gel sensor, a stretchable ion-gel sensor that may have a simple structure and that may simply change a stretching reactivity in various directions by adjusting a content of metal particles may be prepared.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic cross-sectional view of a stretchable ion-gel sensor according to an embodiment;

FIG. 2A is a flowchart illustrating a method of preparing a stretchable ion-gel sensor according to an embodiment;

FIG. 2B is a flowchart illustrating, in detail, a step of separately preparing a lower electrode layer and an upper electrode layer in the method of FIG. 2A;

FIG. 3 is a tilted scanning electron microscopy (SEM) image of a double-layer composite electrode of a stretchable ion-gel sensor according to an embodiment;

FIG. 4 illustrates Bode plots when using different upper layer electrodes (Φ_{Ag}=0.86, 0.75, 0.50) at different strains (ε=0, 30, 50%) of a stretchable ion-gel sensor according to an embodiment;

FIGS. 5A to 5F illustrate effects of controlling a surface area of a first metal particle/polymer layer and a second metal particle/polymer layer depending on a content of silver flakes of a stretchable ion-gel sensor according to an embodiment; and

FIGS. 6A, 6B, and 6C illustrate SEM images of a stretchable ion-gel sensor according to an embedment depending on a content of silver flakes, and FIGS. 6D, 6E, 6F, 6G, 6H, and 6I illustrate atomic force microscopy (AFM) images of the stretchable ion-gel sensor.

### DETAILED DESCRIPTION

Hereinafter, embodiments will be described in detail with reference to the accompanying drawings. However, various alterations and modifications may be made to the embodiments. Here, the embodiments are not meant to be limited by the descriptions of the present disclosure. The embodiments should be understood to include all changes, equivalents, and replacements within the idea and the technical scope of the disclosure.

The terminology used herein is for the purpose of describing particular embodiments only and is not intended to be limiting. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, components, and/or groups thereof but do not preclude the presence or addition of one or more other features, integers, steps, operations, elements, components, and/or groups thereof.

Unless otherwise defined, all terms including technical and scientific terms used herein have the same meaning as commonly understood by one of ordinary skill in the art to which the embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

In addition, when describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

In addition, the terms first, second, A, B, (a), and (b) may be used to describe components of the embodiments. These terms are used only for the purpose of discriminating one component from another component, and the nature, the sequences, or the orders of the components are not limited by the terms.

Components included in one embodiment and components having a common function will be described using the same names in other embodiments. Unless otherwise mentioned, the descriptions on the embodiments may be applicable to the following embodiments and thus, duplicated descriptions will be omitted for conciseness.

Hereinafter, a stretchable ion-gel sensor, and a method of preparing the stretchable ion-gel sensor according to embodiments will be described in detail with reference to embodiments and drawings. However, the present disclosure is not limited to the embodiments and drawings.

A stretchable ion-gel sensor according to an embodiment may include an ion-gel layer, an upper electrode layer disposed on a top surface of the ion-gel layer, and a lower electrode layer disposed on a bottom surface of the ion-gel layer. Each of the upper electrode layer and the lower electrode layer may include a first metal particle/polymer layer, and a second metal particle/polymer layer.

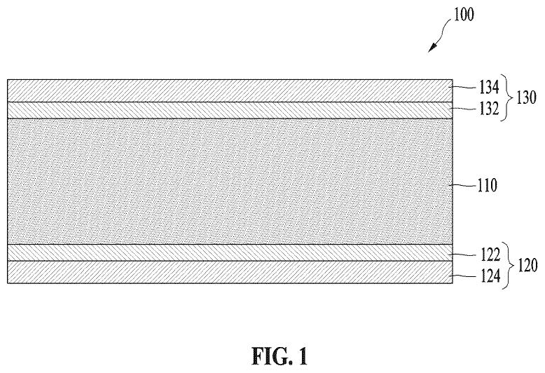

FIG. 1 is a schematic cross-sectional view of a stretchable ion-gel sensor according to an embodiment.

Referring to FIG. 1, a stretchable ion-gel sensor 100 according to an embodiment includes an ion-gel layer 110, a lower electrode layer 120, and an upper electrode layer 130. In an embodiment, the upper electrode layer and the lower electrode layer may be mirror-symmetric to each other with respect to the ion-gel layer.

The ion-gel layer 110 may be interposed between the upper electrode layer 130 and the lower electrode layer 120 such that the upper electrode layer 130 and the lower electrode layer 120 may face each other. Accordingly, a first metal particle/polymer layer 122 and a second metal particle/polymer layer 124 of the lower electrode layer 120, and a first metal particle/polymer layer 132 and a second metal particle/polymer layer 134 of the upper electrode layer 130 may be symmetric to each other with respect to the ion-gel layer 110.

In an embodiment, the ion-gel layer 110 may include a mixture of an ionic liquid and a polymer binder. The ionic liquid may be excellent in a chemical stability and may have a broad electrochemical window. The ionic liquid may include cations and anions.

In an embodiment, the ionic liquid may include at least one of 1-ethyl-3-methylimidazolium bis(trifluoromethylsulfonyl)imide ([EMIM][TFSI]), 1-butyl-3-methylimidazolium bis(trifluoromethylsulfonyl)imide ([BMIM][TFSI]), 1-butyl-3-methylimidazolium hexafluorophosphate ([BMIM][PF₆]), 1-ethyl-3-methylimidazolium tetrafluoroborate ([EMIM][BF₄]), 1-butyl-3-methylimidazolium tetrafluoroborate ([BMIM][BF₄]), 1-butyl-1-methylpyrrolidinium bis(trifluoromethylsulfonyl)imide ([BMPYR][TFSI]), 1-butyl-1-methylpyrrolidinium tris(pentafluoroethyl)trifluorophosphate ([BMPYR][FAP]), 1-ethyl-3-methylimidazolium tris(pentafluoroethyl)trifluorophosphate ([EMIM][FAP]), 1-ethyl-3-methylimidazolium bis(fluorosulfonyl)imide ([EMIM][FSI]), and ethyl-dimethyl-propylammonium bis(trifluoromethylsulfonyl)imide ([EDMPA][TFSI]).

Desirably, the ionic liquid may be 1-butyl-3-methylimidazolium bis(trifluoromethylsulfonyl)imide ([BMIM][TFSI]).

The polymer binder may include an ultraviolet ray (UV)-curable polymer that is cured by UV rays. In this case, the polymer binder may be cured as a predetermined photoinitiator is activated by UV rays.

The polymer binder may also include a block copolymer in addition to the UV-curable polymer. The block copolymer may be, for example, a triblock copolymer.

In an embodiment, the polymer binder may include at least one of poly(vinylidene fluoride-co-hexafluoropropylene) (P(VDF-HFP)), poly(styrene-block-ethylene oxide-block-styrene (PS-PEO-PS), and poly(styrene-block-methyl methacrylate-block-styrene (PS-PMMA-PS).

The polymer binder may be poly(vinylidene fluoride-co-hexafluoropropylene) (P(VDF-HFP)).

By mixing the ionic liquid and the polymer binder and inducing crosslinking of a binder, a gel-type material including an ionic liquid between crosslinked polymers, that is, an ion-gel, may be obtained. The above ion-gel may have a relatively high dielectric constant. For example, the ion-gel may have a dielectric constant of about “10” or greater.

Accordingly, the ion-gel may have transparency in addition to flexibility and/or stretchability.

In an embodiment, the first metal particle and the second metal particle may each have a diameter of 5 μm to 20 μm; 5 μm to 15 μm; 5 μm to 10 μm; 10 μm to 20 μm; 10 μm to 15 μm; or 15 μm to 20 μm.

When the diameter of the first metal particle and the second metal particle is less than 5 μm, it may be difficult to electrically connect the first metal particle and the second metal particle to a metal layer. When the diameter exceeds 20 μm, the thickness of the first metal particle/polymer layer and the second metal particle/polymer layer may increase.

In an embodiment, the first metal particle and the second metal particle may each include at least one of gold (Au), silver (Ag), platinum (Pt), palladium (Pd), copper (Cu), cobalt (Co), zirconium (Zr), zinc (Zn), titanium (Ti), and tin (Sn).

Desirably, the first metal particle and the second metal particle may be silver (Ag) particles.

In an embodiment, the first metal particle of the upper electrode layer 130 may be in an amount of 75% by weight (wt %) to 80 wt %; or 75 wt % to 78 wt % in the first metal particle/polymer layer.

When the amount of the first metal particle of the upper electrode layer 130 is less than 75 wt % in the first metal particle/polymer layer, an ionic resistance may increase due to stretching, which may have an influence on an electrical signal. When the amount of the first metal particle exceeds 80 wt %, the ionic resistance may decrease due to stretching, and an electrode area may uniformly increase during stretching, which may cause a problem in that a stretching reaction is fixed in one direction.

In an embodiment, the second metal particle of the upper electrode layer 130 may be in an amount of 67 wt % to 80 wt %; 67 wt % to 75 wt %; 67 wt % to 70 wt %; 70 wt % to 80 wt %; 70 wt % to 75 wt %; or 75 wt % to 80 wt % in the second metal particle/polymer layer.

When the amount of the second metal particle of the upper electrode layer 130 is less than 67 wt % in the second metal particle/polymer layer, an electrode resistance may increase due to stretching, which may have an influence on an electrical signal. When the amount of the second metal particle exceeds 80 wt %, a problem in that a corresponding electrode is not properly cured may occur.

In an embodiment, the first metal particle of the lower electrode layer 120 may be in an amount of 75 wt % to 80 wt %; or 75 wt % to 78 wt % in the first metal particle/polymer layer.

When the amount of the first metal particle of the lower electrode layer 120 is less than 75 wt % in the first metal particle/polymer layer, the ionic resistance may increase due to stretching, which may have an influence on an electrical signal. When the amount of the first metal particle exceeds 80 wt %, the ionic resistance may decrease due to stretching, and an electrode area may uniformly increase during stretching, which may cause a problem in that a stretching reaction is fixed in one direction.

In an embodiment, the second metal particle of the lower electrode layer 120 may be in an amount of 67 wt % to 80 wt %; 67 wt % to 75 wt %; 67 wt % to 70 wt %; 70 wt % to 80 wt %; 70 wt % to 75 wt %; or 75 wt % to 80 wt % in the second metal particle/polymer layer.

When the amount of the second metal particle of the lower electrode layer 120 is less than 67 wt % in the second metal particle/polymer layer, the electrode resistance may increase due to stretching, which may have an influence on an electrical signal. When the amount of the second metal particle exceeds 80 wt %, a problem in that a corresponding electrode is not properly cured may occur.

When the first metal particle and the second metal particle satisfy the above ranges, the ionic resistance may hardly change due to the stretching, and accordingly, the reactivity may be ignorable.

In particular, when a sufficiently large number of micrometal particles is included, the lower electrode layer 120 may function to allow a stable electric resistance to be maintained even when an electrode is stretched. Unlike a related art, the stretchable ion-gel sensor according to an embodiment may adjust the stretching reactivity of the entire electrolyte system through a composite electrode with a simple structure.

In an embodiment, the polymer layer of the upper electrode layer and the polymer layer of the lower electrode layer each may include at least one of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

Desirably, a polymer of the first metal particle/polymer layer 122, 132 may be styrene-isoprene-butadiene-styrene (SIBS) block copolymer rubber, and a polymer resin of the second metal particle/polymer layer 124, 134 may be polydimethylsiloxane (PDMS). Since the SIBS block copolymer rubber does not require an annealing process, the numerical range of the content of metal particles may be wide, which may facilitate adjusting of the stretching reactivity. However, due to a lack of crosslinking, the electrode resistance may quickly increase due to stretching. Since the PDMS requires an annealing process, numerical values of the content of metal particles may be limited. However, due to sufficient crosslinking, the electrode resistance may not easily change due to stretching, thereby stably maintaining a resistance of a double layer when the PDMS is connected to the lower electrode layer.

In an embodiment, each of the upper electrode layer and the lower electrode layer may further include an elastic substrate (not shown).

Each of the upper electrode layer 130 and the lower electrode layer 120 may be allowed to be embedded in the elastic substrate.

In an embodiment, the elastic substrate may include at least one of polydimethylsiloxane (PDMS), a fluoroelastomer, a poly(vinylidene fluoride-co-hexafluoropropylene) copolymer, thermosetting polyurethane, silicone, Ecoflex, and Dragon skin.

Desirably, the elastic substrate may be polydimethylsiloxane (PDMS).

In an embodiment, when an amount of metal particles in the first metal particle/polymer layer is less than 75 wt %, the stretchable ion-gel sensor may have an impedance of 10⁶ ohms (Ω) to 10⁷ Ω in a frequency range of 100 hertz (Hz) to 102 Hz. When the amount of the metal particles in the first metal particle/polymer layer is greater than or equal to 75 wt % and less than or equal to 80 wt %, the impedance of 10⁶ Ω to 10⁷ Ω may be maintained in the frequency range of 10⁰ Hz to 10² Hz. When the amount of the metal particles in the first metal particle/polymer layer exceeds 80 wt %, the impedance may be in a range of 10⁶ Ω to 10⁹ Ω in the frequency range of 10⁰ Hz to 10² Hz.

When the amount of metal particles in the first metal particle/polymer layer is less than 75 wt %, the impedance may decrease, and when the amount of the metal particles in the first metal particle/polymer layer is greater than or equal to 75 wt % and less than or equal to 80 wt %, the electrode resistance may be stably maintained even under stretching. When the amount of the metal particles in the first metal particle/polymer layer exceeds 80 wt %, the impedance may increase.

The stretchable ion-gel sensor according to an embodiment may simply change the stretching reactivity in various directions by adjusting a composition ratio of metal particles in the first metal particle/polymer layer and the second metal particle/polymer layer with a simple structure.

The stretchable ion-gel sensor according to an embodiment may be applied to artificial electronic skins compatible with the Internet of Things (IoT), skin-attachable flexible sensors, electrocardiogram monitoring medical devices, smart wearable devices, bioelectrodes for analysis of nerve stimulation, and electronic skins for robots. In addition, the stretchable ion-gel sensor may be applied to artificial electronic skin, skin-attachable medical devices, biosignal analysis devices, and robot skins in the future.

A method of preparing a stretchable ion-gel sensor according to an embodiment may include separately preparing an upper electrode layer and a lower electrode layer, forming an ion-gel layer on each of the upper electrode layer and the lower electrode layer, and arranging the ion-gel layer of the upper electrode layer and the ion-gel layer of the lower electrode layer to face each other and performing annealing.

In an embodiment, the separately preparing of the upper electrode layer and the lower electrode layer may include forming a sacrificial layer on a substrate, forming a first metal particle/polymer layer and a second metal particle/polymer layer after placing a pattern mask on the sacrificial layer, annealing the first metal particle/polymer layer and the second metal particle/polymer layer after removing the pattern mask, forming an elastic substrate by coating the annealed first metal particle/polymer layer and the annealed second metal particle/polymer layer with an elastic polymer and curing the elastic polymer, and separating the substrate by removing the sacrificial layer.

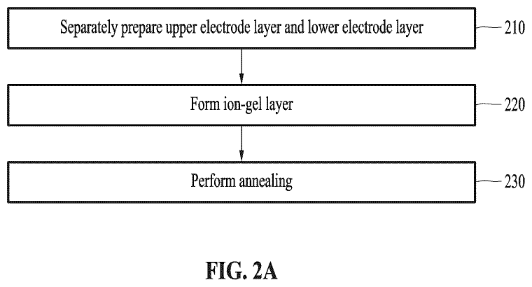

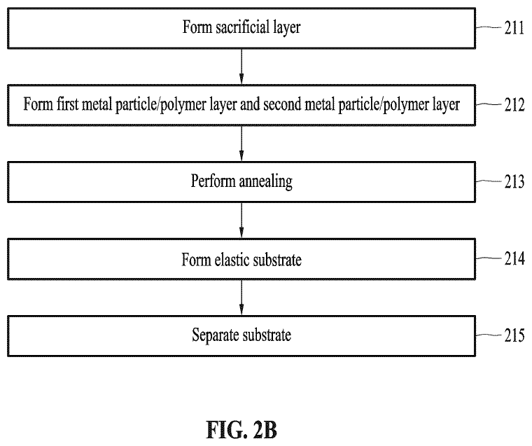

FIG. 2A is a flowchart illustrating a method of preparing a stretchable ion-gel sensor according to an embodiment, and FIG. 2B is a flowchart illustrating, in detail, a step of separately preparing a lower electrode layer and an upper electrode layer in the method of FIG. 2A.

Referring to FIG. 2A, the method may include step 210 of separately preparing an upper electrode layer and a lower electrode layer, step 220 of forming an ion-gel layer, and step 230 of performing annealing.

In step 210, the upper electrode layer and the lower electrode layer may be separately prepared.

Referring to FIG. 2B, step 210 may include step 211 of forming a sacrificial layer, step 212 of forming a first metal particle/polymer layer and a second metal particle/polymer layer, step 213 of performing annealing, step 214 of forming an elastic substrate, and step 215 of separating a substrate.

In step 211, the sacrificial layer may be formed on the substrate.

In an embodiment, the substrate may include at least one of glass, quartz, silicon (Si), silicon oxide (SiO2), polyethylene terephthalate (PET), polyethylene naphthalate (PEN), polycarbonate (PC), polyethersulfone (PES), polyimide (PI), a cyclic olefin copolymer (COC), and polydimethylsiloxane (PDMS).

In an embodiment, the forming of the sacrificial layer on the substrate may include applying at least one solution among polyacrylic acid (PAA), polyvinylpyrrolidone (PVP), dextran, poly(methyl methacrylate) (PMMA), and poly(vinyl alcohol) (PVA) onto the substrate by spin coating, and annealing the substrate at a temperature of 80° C. to 150° C.; 80° C. to 130° C.; 80° C. to 110° C.; 80° C. to 90° C.; 100° C. to 150° C.; 100° C. to 130° C.; 100° C. to 110° C.; or 130° C. to 150° C. for 10 minutes to 60 minutes; 10 minutes to 40 minutes; 10 minutes to 20 minutes; 20 minutes to 60 minutes; 20 minutes to 40 minutes; or 40 minutes to 60 minutes, to form the sacrificial layer.

Desirably, polyacrylic acid (PAA) may be formed as a sacrificial layer at a temperature of 100° C. to 130° C. for 20 minutes.

In step 212, a pattern mask may be placed on the sacrificial layer, and then the first metal particle/polymer layer and the second metal particle/polymer layer may be formed.

In an embodiment, the forming of the first metal particle/polymer layer and the second metal particle/polymer layer may include forming the first metal particle/polymer layer by applying a first metal/polymer resin ink, obtained by mixing a first metal particle and a polymer resin, by blade coating, plasma-treating a surface of the first metal particle/polymer layer, and forming the second metal particle/polymer layer by applying a second metal/polymer resin ink, obtained by mixing a second metal particle and a polymer resin, onto the plasma-treated surface of the first metal particle/polymer layer by blade coating.

In an embodiment, the first metal particle and the second metal particle may each include at least one of gold (Au), silver (Ag), platinum (Pt), palladium (Pd), copper (Cu), cobalt (Co), zirconium (Zr), zinc (Zn), titanium (Ti), and tin (Sn).

Desirably, the first metal particle and the second metal particle may be silver (Ag) particles.

In an embodiment, each of the polymer resins may include at least one of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

The polymer resin of the first metal particle/polymer layer may be polydimethylsiloxane (PDMS), and the polymer resin of the second metal particle/polymer layer may be styrene-isoprene-butadiene-styrene (SIBS) block copolymer rubber.

The plasma-treating of the surface may be performed to generate an ultra-homogeneous and ultra-thin second metal particle/polymer layer on the first metal particle/polymer layer.

In an embodiment, the plasma-treating of the surface may include performing an oxygen plasma treatment in a power range of 80 watts (W) to 120 W; 80 W to 110 W; 80 W to 100 W; 80 W to 90 W; 90 W to 120 W; 90 W to 110 W; 90 W to 100 W; 100 W to 120 W; 100 W to 110 W; or 110 W to 120 W at an flow rate of 30 standard cubic centimeters per minute (sccm) to 50 sccm for 1 second to 300 seconds; 1 second to 250 seconds; 1 second to 200 seconds; 1 second to 150 seconds; 1 second to 100 seconds; 1 second to 50 seconds; 60 seconds to 300 seconds; 60 seconds to 250 seconds; 60 seconds to 200 seconds; 60 seconds to 150 seconds; 60 seconds to 100 seconds; 100 seconds to 300 seconds; 100 seconds to 250 seconds; 100 seconds to 200 seconds; 100 seconds to 150 seconds;

150 seconds to 300 seconds; 150 seconds to 250 seconds; 150 seconds to 200 seconds; 200 seconds to 300 seconds; or 200 seconds to 300 seconds.

When a plasma treatment according to an embodiment is performed, a remarkably excellent and ultra-homogeneous first metal particles/polymer layer and second metal particles/polymer layer may be obtained.

In step 213, the pattern mask may be removed, and then the first metal particle/polymer layer and the second metal particle/polymer layer may be annealed.

In an embodiment, the annealing of the first metal particle/polymer layer and the second metal particle/polymer layer may include performing annealing under a vacuum condition in a temperature range of 100° C. to 200° C.; 100° C. to 180° C.; 100° C. to 150° C.; 100° C. to 130° C.; 130° C. to 200° C.; 130° C. to 180° C.; 130° C. to 150° C.; 150° C. to 200° C.; 150° C. to 180° C.; or 180° C. to 200° C. for 1 hour to 6 hours; 1 hour to 4 hours; 1 hour to 2 hours; 2 hours to 6 hours; 2 hours to 4 hours; or 4 hours to 6 hours.

In step 214, an elastic polymer may be applied onto the annealed first metal particle/polymer layer and the annealed second metal particle/polymer layer and may be cured, to form the elastic substrate.

In an embodiment, the elastic polymer may include at least one of polydimethylsiloxane (PDMS), a fluoroelastomer, a poly(vinylidene fluoride-co-hexafluoropropylene) copolymer, thermosetting polyurethane, silicone, Ecoflex, and Dragon skin.

Desirably, the elastic polymer may be polydimethylsiloxane (PDMS).

In an embodiment, the forming of the elastic substrate may include applying the elastic polymer onto the first metal particle/polymer layer and the second metal particle/polymer layer by spin coating, and performing curing in a temperature range of 20° C. to 100° C.; 20° C. to 80° C.; 20° C. to 60° C.; 20° C. to 40° C.; 40° C. to 100° C.; 40° C. to 80° C.; 40° C. to 60° C.; 60° C. to 100° C.; 60° C. to 80° C.; or 80° C. to 100° C. for 30 minutes to 300 minutes; 30 minutes to 240 minutes; 30 minutes to 180 minutes; 30 minutes to 60 minutes; 60 minutes to 300 minutes; 60 minutes to 240 minutes; 60 minutes to 180 minutes; 120 minutes to 300 minutes; 120 minutes to 240 minutes; 120 minutes to 180 minutes; 180 minutes to 300 minutes; or 180 minutes to 240 minutes.

In step 215, the sacrificial layer may be removed, and the substrate, on which the elastic substrate, the first metal particle/polymer layer, and the second metal particle/polymer layer are formed, may be separated.

In an embodiment, the separating of the substrate by removing the sacrificial layer may include immersing the substate in deionized water in a temperature range of 20° C. to 100° C.; 20° C. to 80° C.; 20° C. to 60° C.; 20° C. to 40° C.; 40° C. to 100° C.; 40° C. to 80° C.; 40° C. to 60° C.; 60° C. to 100° C.; 60° C. to 80° C.; or 80° C. to 100° C. for 30 minutes to 300 minutes; 30 minutes to 200 minutes; 30 minutes to 100 minutes; 30 minutes to 60 minutes; 60 minutes to 300 minutes; 60 minutes to 200 minutes; 60 minutes to 100 minutes; 120 minutes to 300 minutes; 120 minutes to 200 minutes; 180 minutes to 300 minutes; 180 minutes to 200 minutes; or 200 minutes to 300 minutes, and removing the sacrificial layer, to separate the substrate.

As described above, each of the upper electrode layer and the lower electrode layer may be formed.

In step 220, a mixture of an ionic liquid and a polymer binder may be included in each of the upper electrode layer and the lower electrode layer and may be cured, to form the ion-gel layer.

In an embodiment, the ionic liquid may include at least one of 1-ethyl-3-methylimidazolium bis(trifluoromethylsulfonyl)imide ([EMIM][TFSI]), 1-butyl-3-methylimidazolium bis(trifluoromethylsulfonyl)imide ([BMIM][TFSI]), 1-butyl-3-methylimidazolium hexafluorophosphate ([BMIM][PF₆]), 1-ethyl-3-methylimidazolium tetrafluoroborate ([EMIM][BF₄]), 1-butyl-3-methylimidazolium tetrafluoroborate ([BMIM][BF₄]), 1-butyl-1-methylpyrrolidinium bis(trifluoromethylsulfonyl)imide ([BMPYR][TFSI]), 1-butyl-1-methylpyrrolidinium tris(pentafluoroethyl)trifluorophosphate ([BMPYR][FAP]), 1-ethyl-3-methylimidazolium tris(pentafluoroethyl)trifluorophosphate ([EMIM][FAP]), 1-ethyl-3-methylimidazolium bis(fluorosulfonyl)imide ([EMIM][FSI]), and ethyl-dimethyl-propylammonium bis(trifluoromethylsulfonyl)imide ([EDMPA][TFSI]).

In an embodiment, the polymer binder may include at least one of poly(vinylidene fluoride-co-hexafluoropropylene) (P(VDF-HFP)), poly(styrene-block-ethylene oxide-block-styrene (PS-PEO-PS), and poly(styrene-block-methyl methacrylate-block-styrene (PS-PMMA-PS).

A mixing weight ratio of the ionic liquid and the polymer binder may be in a range of 1:9 to 3:7. When the mixing weight ratio of the ionic liquid and the polymer binder is less than 1:9, a range of the content of metal particles that determines the stretching reactivity may change. When the mixing weight ratio of the ionic liquid and the polymer binder exceeds 3:7, excess ionic liquid may be smeared or leak when an ion-gel layer is formed.

Spin coating may be performed on the first metal particle/polymer layer of each of the upper electrode layer and the lower electrode layer, in which the mixture of the ionic liquid and the polymer binder is prepared, at a speed of 200 revolutions per minute (rpm) to 500 rpm; 200 rpm to 400 rpm; 200 rpm to 300 rpm; 300 rpm to 500 rpm; 300 rpm to 400 rpm; or 400 rpm to 500 rpm, for 10 seconds to 60 seconds; 10 seconds to 40 seconds; 10 seconds to 20 seconds; 20 seconds to 60 seconds; 20 seconds to 40 seconds; or 40 seconds to 60 seconds, annealing may be performed at 120° C. for 20 minutes, and a remaining solvent may be dried, to form the ion-gel layer.

In step 230, the ion-gel layer of the upper electrode layer and the ion-gel layer of the lower electrode layer may be arranged to face each other, and annealing may be performed.

The ion-gel layer may be sandwiched between the upper electrode layer and the lower electrode layer such that the upper electrode layer and the lower electrode layer may be mirror-symmetric to each other with respect to the ion-gel layer.

The annealing may be performed at a temperature of 80° C. to 150° C.; 80° C. to 130° C.; 80° C. to 100° C.; 100° C. to 150° C.; 100° C. to 130° C.; or 130° C. to 150° C. for 1 minute to 60 minutes; 1 minute to 40 minutes; 1 minute to 20 minutes; 1 minute to 10 minutes; 10 minutes to 60 minutes; 10 minutes to 40 minutes; 10 minutes to 20 minutes; 20 minutes to 60 minutes; 20 minutes to 40 minutes; or 40 minutes to 60 minutes, to prepare a stretchable ion-gel sensor according to an embodiment.

According to embodiments, by the method of preparing the stretchable ion-gel sensor according to an embodiment, a stretchable ion-gel sensor that may have a simple structure and that may simply change a stretching reactivity in various directions by adjusting a content of metal particles may be prepared.

Hereinafter, the present disclosure will be described in detail with reference to the following examples. However, the technical idea of the present invention is not limited or restricted thereby.

### EXAMPLE

A polyacrylic acid (PAA) solution was formed by dissolving 5 wt % of PAA in water, was applied onto a glass substrate by spin coating (1000 rpm, 30 seconds), and then annealing was performed at 120° C. for 20 minutes, to form a sacrificial layer for transferring an electrode film.

Silver (Ag) particles (diameter=8 to 10 μm) were mixed with a SIBS solution (20 wt % in toluene) to prepare Ag/SIBS ink (a proportion of silver to SIBS=1, 2, 3, 4, 5, 6) for an upper layer electrode under various different conditions. Ag particles were mixed with a PDMS solution (33 wt % in MEK) to prepare Ag/PDMS ink (a proportion of silver to PDMS=3) for a lower layer electrode.

Subsequently, a pattern mask was placed on the glass substrate coated with PAA, the Ag/SIBS ink was first applied by blade coating, and the surface was treated with oxygen plasma (100 W, 1 minute). After the Ag/PDMS ink was also applied onto the surface by blade coating, the pattern mask was peeled off and annealing was performed under vacuum conditions at 150° C. for 2 hours.

Spin coating was performed with PDMS, which is to function as a stretchable substrate, (350 rpm, 30 seconds) and annealing was performed at 80° C. for 2 hours. The above film coated on the glass substrate was immersed in water at 80° C., and after 1 hour, a PAA sacrificial layer was dissolved, to prepare a lower electrode layer and an upper electrode layer onto which an electrode film including a first metal particle/polymer layer and a second metal particle/polymer layer was transferred.

Subsequently, ionic liquid (BMIM:TFSI) was mixed with a stretchable polymer e-PVDF-HFP solution (20 wt % in MEK), to prepare an ion-gel solution (a mass ratio of the ionic liquid to the polymer was 1:9). The ion-gel solution was applied onto a prepared composite electrode line by spin coating (300 rpm, 30 seconds), and annealing was performed at 120° C. for 20 minutes to dry a remaining solvent.

An ion-gel layer of the lower electrode layer and an ion-gel layer of the upper electrode layer were sandwiched such that surfaces of the ion-gel layers face each other, and annealing was performed at 120° C. for 20 minutes, to prepare a stretchable ion-gel sensor.

A response of an ion-gel sensor to an external strain may be significantly affected by an effective surface area of a composite electrode of the stretchable ion-gel sensor. The composite electrode in an embodiment may include two composite layers. A lower composite layer may have a stable permeable network of conductive fillers, while an upper composite layer may have a variable amount of fillers. Since fillers in an upper layer are connected to a stable percolation network in a lower layer, electrical performance of a double-layer composite may be less susceptible to mechanical stretching. The fillers in the upper layer may be partially exposed to the air. Since the air-exposed fillers effectively contribute to an electrochemical interaction with an electrolyte, a change of an impedance spectrum under stretching may be governed by an effective fraction of the air-exposed fillers.

An ion-gel sensor with a top-bottom structure may be electrically characterized using an equivalent circuit and a Bode plot. A flat impedance in a middle frequency region of the Bode plot may represent a bulk ionic resistance (Rion) that is determined by geometric factors such as an electrode surface area (A) and a distance (d) between electrodes for a given ion conductivity (σ).

A PDMS composite and a thermoplastic polymer composite may be representative stretchable electrodes, and a model study with a double-layer composite electrode having an Ag flake/PDMS composite as a lower layer and an Ag flake/polystyrene-block-poly(isobutylene)-block-polystyrene (SIBS) composite as an upper layer was investigated.

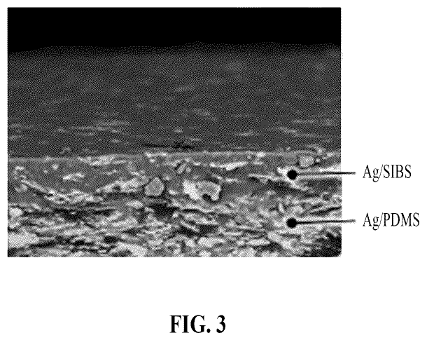

FIG. 3 is a tilted scanning electron microscopy (SEM) image of a double-layer composite electrode of a stretchable ion-gel sensor according to an embodiment. A fraction of Ag flakes in a PDMS composite was commonly used as Φ_{Ag,PDMS}=0.75, which was the same as a lower layer. To adjust an effective surface area of a filler, a fraction of Ag flakes in an upper layer, φAg ≡W_{Ag}/(W_{Ag}+W_{SIBS}), was changed (Φ_{Ag}=0.86, 0.83, 0.80, 0.75, 0.67, 0.50).

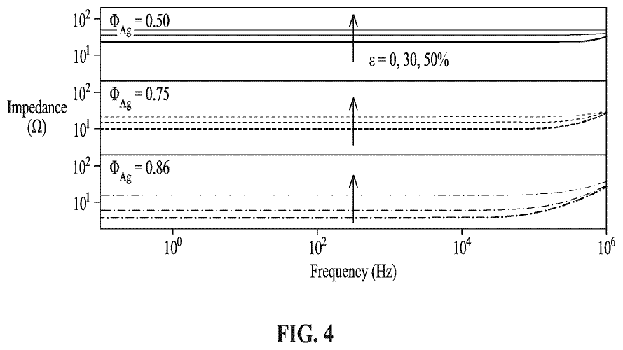

FIG. 4 illustrates Bode plots when using different upper layer electrodes (Φ_{Ag}=0.86, 0.75, 0.50) at different strains (ε=0, 30, 50%) of a stretchable ion-gel sensor according to an embodiment.

FIG. 4 illustrates an impedance profile of a double-layer composite electrode in the absence of an ion gel, depending on Φ_{Ag} and elongational strain (ε=0, 30, 50%) in the entire frequency range (10⁻¹ to 10⁶ Hz). An impedance was less than 10² Ω in the entire frequency range and a plateau line was maintained up to 10⁵ Hz, which is typical for electrical conductors. Since an impedance in the presence of the ion gel is greater than 10⁴ Ω in the entire frequency range, an electrode does not have an influence on an impedance profile of an ion-gel sensor.

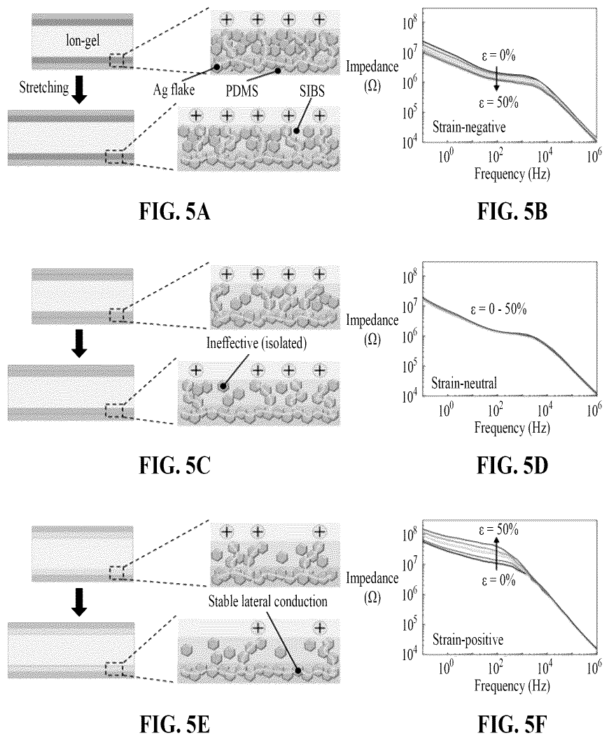

FIGS. 5A to 5F illustrate effects of controlling a surface area of a first metal particle/polymer layer and a second metal particle/polymer layer depending on a content of silver flakes of a stretchable ion-gel sensor according to an embodiment.

Referring to FIGS. 5A and 5B, it can be confirmed that, when a content of a first metal particle is 86 wt %, an effective surface area increased due to a sufficiently large number of connections between metal particles due to stretching, and the ionic resistance gradually decreased. Referring to FIGS. 5C and 5D, it can be confirmed that, when the content of the first metal particle is 75 wt %, the effective surface area was maintained by balancing a disconnection between metal particles due to stretching and an increase in the surface area, and thus, almost no ionic resistance reactivity was observed. Referring to FIGS. 5E and 5F, it can be confirmed that, when the content of the first metal particle is 50 wt %, the effective surface area was greatly reduced due to a dominant influence of a disconnection between metal particles due to stretching, and the ionic resistance gradually increased.

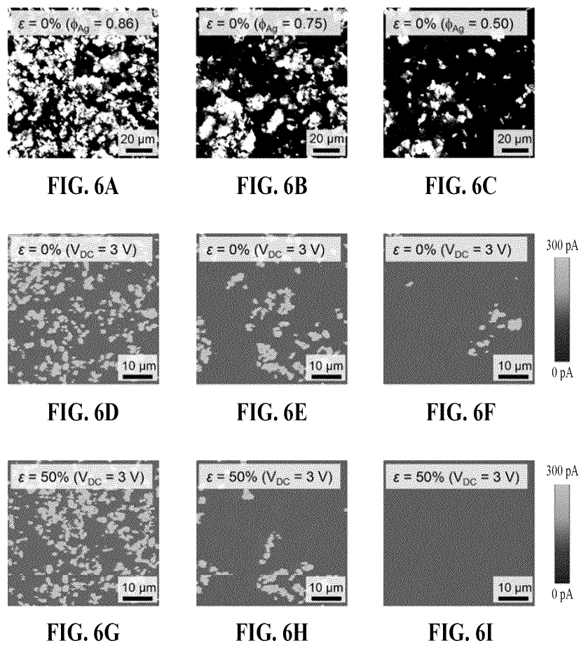

FIGS. 6A, 6B, and 6C illustrate SEM images of a stretchable ion-gel sensor according to an embedment depending on a content of silver flakes, and FIGS. 6D, 6E, 6F, 6G, 6H, and 6I illustrate atomic force microscopy (AFM) images of the stretchable ion-gel sensor.

Referring to FIGS. 6A to 6I, it can be confirmed that, when the SEM images of FIGS. 6A to 6C and the AFM images of FIGS. 6D to 6F at an elongational strain of 0% are compared, only some of silver flakes near a surface contribute to an effective surface area. It can be confirmed that, when the AFM images of FIGS. 6D to 6F at the elongational strain of 0% are compared to the AFM images of FIGS. 6G to 6I at an elongational strain of 50%, the effective surface area increased as a content of a first metal particle is 86 wt % in FIGS. 6D and 6G. Referring to FIGS. 6E and 6H, it can be confirmed that the effective surface area was similarly maintained as the content of the first metal particle is 75 wt %. Lastly, referring to FIGS. 6F and 6I, it can be confirmed that most of the effective surface area was lost as the content of the first metal particle is 50 wt %.

The stretchable ion-gel sensor according to an embodiment may have a simple structure to provide a stretching insensitivity, and it is confirmed that the stretchable ion-gel sensor stably operates even when stretching stimulation is applied 1,000 times or more.

An influence of an electrically effective surface area of the stretchable ion-gel sensor according to an embodiment on a strain response was investigated, and facile designs of composite electrodes were demonstrated to obtain strain-negative, strain-neutral, and strain-positive impedance responses to external stretching. By controlling a fraction of conductive fillers in a composite electrode, the electrically effective surface area may be adjusted. It was revealed that a composite with a large fraction of conductive fillers is strain-negative in impedance, whereas a composite with a small fraction of fillers is strain-positive, and a composite with a moderate fraction of fillers may be strain-neutral. The above result is dependent on whether an electrical connection to a surface is to be decreased (strain-positive), maintained (strain-neutral), and increased (strain-negative) under stretching. Embodiments provide a practical strategy to control a strain response of the stretchable ion-gel sensor.

While the embodiments are described, it will be apparent to one of ordinary skill in the art that various alterations and modifications in form and details may be made in these embodiments without departing from the spirit and scope of the claims and their equivalents. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents to the claims are also within the scope of the following claims.

What is claimed is:

1. A stretchable ion-gel sensor comprising:
an ion-gel layer;
an upper electrode layer disposed on a top surface of the ion-gel layer; and
a lower electrode layer disposed on a bottom surface of the ion-gel layer,
wherein each of the upper electrode layer and the lower electrode layer comprises a first metal particle/polymer layer, and a second metal particle/polymer layer.

2. The stretchable ion-gel sensor of claim 1, wherein
the ion-gel layer comprises a mixture of an ionic liquid and a polymer binder,
the ionic liquid comprises at least one selected from a group consisting of 1-ethyl-3-methylimidazolium bis(trifluoromethylsulfonyl)imide ([EMIM][TFSI]), 1-butyl-3-methylimidazolium bis(trifluoromethylsulfonyl)imide ([BMIM][TFSI]), 1-butyl-3-methylimidazolium hexafluorophosphate ([BMIM][PF₆]), 1-ethyl-3-methylimidazolium tetrafluoroborate ([EMIM][BF₄]), 1-butyl-3-methylimidazolium tetrafluoroborate ([BMIM][BF₄]), 1-butyl-1-methylpyrrolidinium bis(trifluoromethylsulfonyl)imide ([BMPYR][TFSI]), 1-butyl-1-methylpyrrolidinium tris(pentafluoroethyl)trifluorophosphate ([BMPYR][FAP]), 1-ethyl-3-methylimidazolium tris(pentafluoroethyl)trifluorophosphate ([EMIM][FAP]), 1-ethyl-3-methylimidazolium bis(fluorosulfonyl)imide ([EMIM][FSI]), and ethyl-dimethyl-propylammonium bis(trifluoromethylsulfonyl)imide ([EDMPA][TFSI]), and
the polymer binder comprises at least one selected from a group consisting of poly(vinylidene fluoride-co-hexafluoropropylene) (P(VDF-HFP)), poly(styrene-block-ethylene oxide-block-styrene (PS-PEO-PS), and poly(styrene-block-methyl methacrylate-block-styrene (PS-PMMA-PS).

3. The stretchable ion-gel sensor of claim 1, wherein
the first metal particle and the second metal particle each have a diameter of 5 micrometers (μm) to 20 μm, and
each of the first metal particle and the second metal particle comprises at least one selected from a group consisting of gold (Au), silver (Ag), platinum (Pt), palladium (Pd), copper (Cu), cobalt (Co), zirconium (Zr), zinc (Zn), titanium (Ti), and tin (Sn).

4. The stretchable ion-gel sensor of claim 1, wherein
the first metal particle of the upper electrode layer is in an amount of 75% by weight (wt %) to 80 wt % in the first metal particle/polymer layer, and
the second metal particle of the upper electrode layer is in an amount of 67 wt % to 80 wt % in the second metal particle/polymer layer.

5. The stretchable ion-gel sensor of claim 1, wherein
the first metal particle of the lower electrode layer is in an amount of 75 wt % to 80 wt % in the first metal particle/polymer layer, and
the second metal particle of the lower electrode layer is in an amount of 67 wt % to 80 wt % in the second metal particle/polymer layer.

6. The stretchable ion-gel sensor of claim 1, wherein the polymer layer of the upper electrode layer and the polymer layer of the lower electrode layer each comprise at least one selected from a group consisting of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

7. The stretchable ion-gel sensor of claim 1, wherein
each of the upper electrode layer and the lower electrode layer further comprises an elastic substrate, and
the elastic substrate comprises at least one selected from a group consisting of polydimethylsiloxane (PDMS), a fluoroelastomer, a poly(vinylidene fluoride-co-hexafluoropropylene) copolymer, thermosetting polyurethane, silicone, Ecoflex, and Dragon skin.

8. The stretchable ion-gel sensor of claim 1, wherein
when an amount of metal particles in the first metal particle/polymer layer is less than 75 wt %, the stretchable ion-gel sensor has an impedance of 10⁶ ohms (Ω) to 10⁷ Ω in a frequency range of 10⁰ hertz (Hz) to 10² Hz,
when the amount of the metal particles in the first metal particle/polymer layer is greater than or equal to 75 wt % and less than or equal to 80 wt %, the impedance of 10⁶ Ω to 10⁷ Ω is maintained in the frequency range of 10⁰ Hz to 10² Hz, and
when the amount of the metal particles in the first metal particle/polymer layer exceeds 80 wt %, the impedance is in a range of 10⁶ Ω to 10⁹ Ω in the frequency range of 10⁰ Hz to 10² Hz.

9. A method of preparing a stretchable ion-gel sensor, the method comprising:
separately preparing an upper electrode layer and a lower electrode layer;
forming an ion-gel layer on each of the upper electrode layer and the lower electrode layer; and
arranging the ion-gel layer of the upper electrode layer and the ion-gel layer of the lower electrode layer to face each other and performing annealing.

10. The method of claim 9, wherein the separately preparing of the upper electrode layer and the lower electrode layer comprises:
forming a sacrificial layer on a substrate;
forming a first metal particle/polymer layer and a second metal particle/polymer layer after placing a pattern mask on the sacrificial layer;
annealing the first metal particle/polymer layer and the second metal particle/polymer layer after removing the pattern mask;
forming an elastic substrate by coating the annealed first metal particle/polymer layer and the annealed second metal particle/polymer layer with an elastic polymer and curing the elastic polymer; and
separating the substrate by removing the sacrificial layer.

11. The method of claim 10, wherein the forming of the sacrificial layer on the substrate comprises applying at least one solution selected from a group consisting of polyacrylic acid (PAA), polyvinylpyrrolidone (PVP), dextran, poly(methyl methacrylate) (PMMA), and poly(vinyl alcohol) (PVA) onto the substrate by spin coating, and annealing the substrate at a temperature of 80° C. to 150° C. for 10 minutes to 60 minutes, to form the sacrificial layer.

12. The method of claim 10, wherein
each of the first metal particle and the second metal particle comprises at least one selected from a group consisting of gold (Au), silver (Ag), platinum (Pt), palladium (Pd), copper (Cu), cobalt (Co), zirconium (Zr), zinc (Zn), titanium (Ti), and tin (Sn), and
the polymer layer of the upper electrode layer and the polymer layer of the lower electrode layer each comprise at least one selected from a group consisting of polydimethylsiloxane (PDMS), styrene-ethylene-butylene-styrene (SEBS) block copolymer rubber, styrene-isoprene-styrene (SIS) block copolymer rubber, styrene-butadiene-styrene (SBS) block copolymer rubber, polyisoprene rubber, styrene butadiene (SB) block copolymer rubber, styrene-isoprene (SI) block copolymer rubber, styrene-isoprene-butadiene-styrene (SIBS) block copolymer rubber, styrene-ethylene-propylene-styrene (SEPS) block copolymer rubber, and styrene-ethylene-propylene (SEP) block copolymer rubber.

13. The method of claim 10, wherein the forming of the first metal particle/polymer layer and the second metal particle/polymer layer comprises:
forming the first metal particle/polymer layer by applying a first metal/polymer resin ink, obtained by mixing a first metal particle and a polymer resin, by blade coating;
plasma-treating a surface of the first metal particle/polymer layer; and
forming the second metal particle/polymer layer by applying a second metal/polymer resin ink, obtained by mixing a second metal particle and a polymer resin, onto the plasma-treated surface of the first metal particle/polymer layer by blade coating.

14. The method of claim 13, wherein the plasma-treating of the surface comprises performing an oxygen plasma treatment in a power range of 80 watts (W) to 120 W at an oxygen flow rate of 30 standard cubic centimeters per minute (sccm) to 50 sccm for 1 second to 300 seconds.

15. The method of claim 10, wherein the annealing of the first metal particle/polymer layer and the second metal particle/polymer layer comprises performing annealing under a vacuum condition in a temperature range of 100° C. to 200° C. for 1 hour to 6 hours.

16. The method of claim 10, wherein the forming of the elastic substrate comprises applying the elastic polymer onto the first metal particle/polymer layer and the second metal particle/polymer layer by spin coating, and performing annealing in a temperature range of 20° C. to 100° C. for 30 minutes to 300 minutes.

17. The method of claim 10, wherein the separating of the substrate by removing the sacrificial layer comprises immersing the substate in deionized water in a temperature range of 20° C. to 100° C. for 30 minutes to 300 minutes and removing the sacrificial layer, to separate the substrate.

18. The method of claim 9, wherein the arranging of the ion-gel layer of the upper electrode layer and the ion-gel layer of the lower electrode layer to face each other and performing annealing is performed at a temperature of 80° C. to 150° C. for 1 minute to 60 minutes.
